(19) United States
(12) Patent Application Publication
(10) Pub. No.: US 2002/0156434 A1
(43) Pub. Date: Oct. 24, 2002

(54) Stabilizing catheter for protein drug delivery

(75) Inventors: William P. Van Antwerp, Valencia, CA (US); Poonam S. Gulati, La Canada, CA (US)

Correspondence Address: Irvin C. Harrington, III, FOLEY & LARDNER, 35th Floor, 2029 Century Park East, Los Angeles, CA, 90067-3021, US

(73) Assignee: MINIMED INC.

(21) Appl. No.: 10/035,831

(22) Filed: Dec. 28, 2001

Related U.S. Application Data
(63) Continuation of application No. 09/042,138, filed on Mar. 13, 1998, now abandoned.
(63) Continuation of application No. 09/324,783, filed on Jun. 3, 1999, now abandoned.
(60) Provisional application No. 60/317,256, filed on Sep. 5, 2001.

(51) Int. Cl. A61L 2/00; B05D 3/00; A61M 5/32

(52) U.S. Cl. 604/265; 427/2.3; 604/527

(57) ABSTRACT

Stabilizing catheters for delivery of one or more protein drugs to a patient. The stabilizing catheter embodiments of the invention maintain or preserve a biologically/pharmacologically active form of the protein drug for delivery to a site within the body. Particular embodiments include a tubing layered with a hydrophilic and mobile polymer that aids in the maintenance or preservation of an active conformer of the protein drug. These embodiments of the stabilizing catheter prevent site loss of protein drugs, such as insulin. Other embodiments include a tubing that is layered with a material that substantially prevents diffusion of small, insulin formulation-stabilizing molecules out from the catheter, as well as substantially prevents the diffusion of small, insulin formulation-destabilizing molecules into the catheter, during a period of insulin infusion. In effect, these embodiments of the stabilizing catheter maintain the stabilizing effect of a particular insulin formulation, and consequently, substantially prevents occlusions/deposits from being formed during a period set for insulin delivery. Still other embodiments are directed to a combination of these features of the stabilizing catheters of the invention.

### CROSS REFERENCE TO RELATED APPLICATIONS

This application is related to Ser. No. 09/042,138, filed Mar. 13, 1998, which is a continuation application of U.S. patent application Ser. No. 08/742,377, filed Nov. 1, 1996; this application is also related to Ser. No. 09/324,783, filed Jun. 3, 1999. The contents of each of these related applications are incorporated herein by reference in their entireties. This application is also related to copending United States patent application “Barrier Catheter Apparatus and Method,” attorney docket No. 047711/0284, filed concurrently herewith, the contents of which are incorporated by reference herein

### FIELD OF THE INVENTION

This invention relates to protein drug delivery devices and related methods, and in particular embodiments, to catheters for insulin delivery to a site within the body.

### BACKGROUND OF THE INVENTION

Insulin is used for the daily treatment of patients with type 1, and in many cases type 2, diabetes mellitus. Conventionally, insulin is delivered via syringe injections. However, intensive management of Type 1 diabetes can involve the use of insulin pumps.

These insulin pumps are part of infusion systems where insulin is forced from a reservoir, usually to a subcutaneous, intravenous or intraperitoneal site within the body of a patient. The reservoir, which must be replaced or refilled periodically, is either attached to or implanted in the patient. During insulin pump therapy, insulin is retained in the reservoir so that the drug can be delivered to the patient over extended periods of time. The period of time for drug delivery is generally several days for subcutaneous delivery, but can be up to several months for insulin delivered intraperitoneally using an implanted pump.

Regardless of the precise mode of delivery, however, insulin pump therapy requires delivery to sites within the body via a delivery catheter. Moreover, the delivery catheter can affect the delivered insulin, as insulin is inherently unstable when used over the extended periods of time necessary for extended drug delivery in a delivery device. As a consequence, the overall stability of a insulin and insulin formulations are a concern for drug delivery via pumps.

### SUMMARY OF THE DISCLOSURE

Embodiments of the present invention address several problems associated with the delivery of protein drugs via infusion devices, known as pumps. Because most proteins, especially relatively small hormone-like proteins, such as insulin, are inherently unstable, embodiments of the present invention utilize certain, stabilizing materials in the construction of novel stabilizing catheters.

Embodiments of the present invention stabilize protein drugs, such as insulin, delivered via either internally or externally placed catheters against two general classes chemical and/or chemo-physical events. These events include interactions of the protein drugs with the surfaces of the interior walls of a delivery catheter and interactions of protein drugs with the environmental milieu in which the protein is contained, i.e., the protein formulation.

Protein-surface interactions are destabilizing to protein drugs because these interactions generally lead to denaturation of the complex and defined, 3-dimensional protein structure due to the relatively hydrophobic nature of these surfaces. As a consequence, the biological/pharmacological activity of the protein drug is decreased. In these events, the hydrophilic protein drug is destabilized as it contacts the hydrophobic polymeric, metallic, or other material surfaces of a delivery catheter. This phenomenon is related to the generally low free surface energies of these materials, typically on the order of about 40 dyne/cm². At these low free surface energy, protein-based medications can be absorbed quite readily and can denature on the catheter surfaces. This event can lead to sticking of the denatured, or partially denatured, proteins to the surface forming protein deposits and protein aggregates. A further negative consequence of these interactions is that once denaturation and/or aggregation occurs, the protein drug is generally not bio-available to the patient and may in some cases lead to undesired immunological responses. This phenomenon is referred to herein as “site loss,” and is described below.

Interactions of protein-based medications with the environmental milieu in which the protein is contained can also lead to denaturation of the native and biologically/pharmacologically active form of the protein. For example, a problem that can be encountered with implantable protein drug delivery devices is that the integrity of a particular protein formulation can become compromised as the protein formulation is resident in and traverses through a delivery catheter. This problem occurs due to changes in the environmental milieu of the protein drug formulation as it is resident in the delivery catheter. This problem is generally related to the diffusion of small destabilizing molecules into the delivery catheter, as well as diffusion of small stabilizing molecules out from the delivery catheter, prior to protein drug delivery to an appropriate site within the body.

Stabilizing catheter embodiments of the present invention solve these problems by substantially maintaining biologically/pharmacologically active protein drug conformers and/or by maintaining the composition of a particular protein drug formulation as the protein drug and/or protein drug formulation traverses through a lumen of a delivery catheter. These stabilizing catheters perform these functions by providing one or more stabilizing materials to be included in their construction.

According to embodiments of the present invention, stabilizing catheters for protein drug delivery are disclosed. In particular embodiments, the stabilizing catheters are used for implantable or external infusion device insulin therapy. Further, embodiments of these stabilizing catheters are especially useful for external or implantable infusion device therapy which use high concentration insulin formulations of about 100 U/ml or greater. Additionally, these stabilizing catheters are particularly suitable to stabilize insulin and insulin formulations which include monomeric insulin, such as human insulin analogs, like LISPRO insulin or the like.

Embodiments of the stabilizing catheters provide a tubing having one or more of its internal surfaces bearing a hydrophilic coating which substantially maintains a biologically/pharmacologically active form of a protein drug, particularly when the stabilizing catheter is used to deliver complex protein-based medications, such as insulin. Further, hydrophilic coatings used in accordance with embodiments of the invention also should possess a high degree of mobility of the one or more chemical groups that comprise the hydrophilic coating. Accordingly, coatings used in accordance with embodiments of the invention should possess properties that impart a certain degree of hydrophilicity and mobility so that interactions of the protein drug with the interior surfaces of a stabilizing catheter do not substantially denature, or absorb, the protein. As a consequence, the biological/pharmacological activity of the delivered protein-based medication is substantially maintained. A further consequence of the use of a stabilizing catheter in accordance with embodiments of the invention is that since the protein drug is not denatured as it contacts and interacts with the interior surfaces of the stabilizing catheter, undesired protein drug deposits are concomitantly reduced or eliminated. An exemplary hydrophilic and mobile coating material for use in stabilizing catheters of the present invention is any polymeric material containing a relatively high content of polyethylene glycol units. Polyethylene glycol (PEG), or like polymers, possess good hydrophilicity and mobility characteristics such that when a protein drug interacts with a surface coated with PEG the protein drug is not denatured due both to the hydrophilicity as well as its mobility of the polymer. Other hydrophilic polymers are disclosed in related applications, i.e., Ser. No. 09/042,138, filed Mar. 13, 1998, which is a continuation application of U.S. patent application Ser. No. 08/742,377, filed Nov. 1, 1996 and Ser. No. 09/324,783, filed Jun. 3, 1999. The contents of each of these related applications are incorporated by reference in their entireties.

Still other embodiments of stabilizing catheters in accordance with the invention include a tubing with at least one layer that includes materials that substantially reduce diffusion of small molecules through the tubing. Thus, when the stabilizing catheter including the tubing is used for insulin delivery, for example, the insulin formulation, and consequently insulin itself, is stabilized, maintained or preserved as compared with insulin delivered via a different tubing that is substantially free of the stabilizing properties of the embodiments of the stabilizing catheter. This feature of the stabilizing catheter substantially prevents the formation of deposits/occlusions which can impede or block fluid flow during a period set for insulin delivery.

Generally, the stabilizing catheter embodiments of the present invention that provide a diffusion barrier include one or more layers with at least one layer being formed from polytetrafluoroethane, saran (PVOC (polyvinyloenechloride)) polysulfone, glass, a metal, derivatives of these materials, and mixtures of these materials. These materials present a stabilizing layer that substantially reduces diffusion of formulation-stabilizing small molecules out from the catheter, as well as reduces diffusion of formulation-destabilizing small molecules into the catheter. As a result, the stabilizing catheter maintains the integrity of a particular protein drug and protein drug formulation as it moves from a infusion device reservoir to a targeted site of delivery.

In various embodiments of the present invention, the stabilizing catheter may include one or more layers, with a tubing having at least two layers being preferred for an implanted stabilizing catheter. In some particular embodiments which include at least two layers, the outermost layer of the catheter is formed from a layer of a silicone material with an inner layer being formed from a stabilizing material, such as polytetrafluoroethane, saran (PVOC), polysulfone, glass, metal, derivatives of these materials, as well as mixtures of these materials. Other particular embodiments also include an innermost layer comprised of protein drug compatible materials, such as a coating, or layer, which are hydrophilic or which possesses the characteristics of a surfactant. This layer or coating substantially precludes the protein drug contained within the formulation from interacting unfavorably with the walls of the catheter, thus diminishing denaturation, or unfolding, of the protein drug, and concomitantly reduces site loss.

Embodiments of the stabilizing catheter reduce diffusion of neutrally charged molecules, charged molecules, including metal ions, and mixtures of these molecules. Preferred embodiments of the stabilizing catheter substantially reduce diffusion of small molecules having a molecular weight of about 18 g/mole to about 500 g/mole. In particular embodiments, the stabilizing catheter substantially reduces diffusion of neutral molecules, such as phenol and phenolic derivatives, out from the tubing, as well as reduces diffusion of neutral molecules, such as carbon dioxide, into the tubing.

In preferred embodiments, the stabilizing catheter reduces the diffusional flow of carbon dioxide into the tubing by up to about 1000 fold, preferably at least about 10-100 fold and/or decreases the diffusional flow of phenol out from the tubing by up to about 100 fold, preferably at least about 2-20 fold as compared to the diffusional flow of carbon dioxide into and/or phenol out from a different tubing that does not include a stabilizing layer. In certain preferred embodiments, the stabilizing catheter provides a diffusional barrier to phenol, such that the loss of phenol through the tubing is less than about 10%, preferably less than about 5% at an insulin infusion rate of about 20 U/day. When using a stabilizing catheter that includes a layer of Teflon or a layer of Saran as the stabilizing layer, the thickness of either of these layers is about 0.002 in to about 0.02 in (i.e., 5-50 microns).

Other embodiments of the present invention include an infusion system for protein drug delivery which includes an infusion device housing, a reservoir for containing one or more protein drugs and a stabilizing catheter for insulin delivery connected to the reservoir and leading to a delivery site within the body of the patient or a user. The delivery site can be subcutaneous, intravenous and/or intraperitoneal. In the invention, embodiments of the infusion system include a tubing, including at least one layer that is made from materials that reduce diffusion of small molecules through the tubing. When infusion systems including the stabilizing catheter are used to deliver insulin to a patient, insulin is stabilized as compared to insulin delivered via infusion systems which include a different catheter made from materials which substantially permit the diffusion of small molecules through the catheter. Thus, embodiments of the infusion system substantially reduce the formation of deposits/occlusions during insulin delivery, especially when using high concentration insulin formulation and/or monomeric insulin analogs.

Other embodiments of the present invention include methods of stabilizing an protein drug formulation, such as an insulin formulation, while it passes through a stabilizing catheter. These methods include providing a stabilizing catheter, as disclosed above, to a patient so as to stabilize the insulin formulation as it passes through the stabilizing catheter, and flowing a fluid including insulin through the stabilizing catheter. These methods substantially reduce the formation of protein deposits/occlusions during insulin delivery, especially for high concentration insulin formulation and/or monomeric insulin analog formulations.

Further, these methods create a diffusion barrier to small, neutral molecules, charged molecules, including metal ions, and mixtures of these molecules. In particular embodiments, these methods substantially reduce the diffusion of small molecules having a molecular weight of about 18 g/mole to about 300 g/mole through the stabilizing catheter. In certain embodiments, these methods substantially reduce diffusion of neutral molecules, such as phenol and/or phenolic derivatives, out from the stabilizing catheter, as well as reduce diffusion of neutral molecules, such as carbon dioxide, into the stabilizing catheter. As a consequence, the stabilizing catheter stabilizes, maintains or preserves the integrity of a particular an protein drug formulation, particularly an insulin formulation.

Other features and advantages of the invention will become apparent from the following detailed description, taken in conjunction with the accompanying drawings, which illustrate, by way of example, various features of embodiments of the invention.

### BRIEF DESCRIPTION OF THE DRAWINGS

A detailed description of embodiments of the invention will be made with reference to the accompanying drawings, wherein like numerals designate corresponding parts in the several figures.

FIG. 1 is an illustration of a prior art catheter with an insulin/Tris/CO₂ occlusion contained within the lumen of the catheter.

FIG. 2 is a schematic illustration of an embodiment of the stabilizing catheter of the invention that includes a hydrophilic and mobile coating layer.

FIG. 3 is an illustration of the effect of using an embodiment of the stabilizing catheter of the invention, as shown in FIG. 2, in preventing site loss of insulin.

FIG. 4 is a schematic illustration of an embodiment of the stabilizing catheter of the invention that includes a barrier layer.

FIG. 5A is a graph of the change in phenol concentration as a function of time for various conventional catheter/tubing materials.

FIG. 5B show similar results as FIG. 5A, except that the change in phenol concentration as a function of catheter/tubing materials has been adjusted by the amount of phenol loss from a standard reservoir as depicted in FIG. 5A.

FIG. 6 is an graphical illustration of a model of insulin stabilization brought about by maintaining both the phenol content and the pH of a particular insulin formulation.

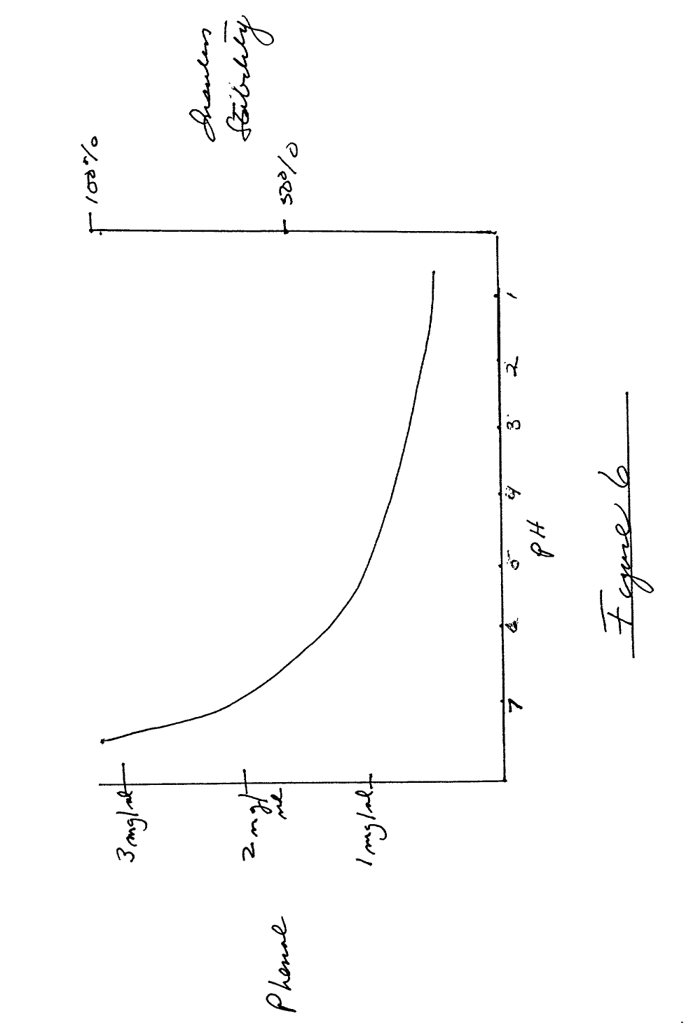

### DETAILED DESCRIPTION OF THE PREFERRED EMBODIMENTS

I. Definitions

For purposes of the present invention, as disclosed and claimed herein, the following terms are defined.

The term “protein drug” or “protein-based medication” encompasses any protein-containing formulation administered to a person to achieve a desired biological/pharmacological effect.

The term “stability” refers to the physical and/or chemical stability of a particular protein and/or protein drug formulation during a period set for protein drug delivery. Generally, for insulin delivery, this period is less than one week for subcutaneous insulin infusion, and less than one year for interperitoneal insulin infusion, more specifically for about 90 days for implantable infusion devices.

A protein drug, such as insulin, is “stabilized”, “maintained”, “preserved” or the like, in embodiments of the present invention if the protein drug is delivered to a desired site within the body with a reduction in “site loss” of the protein drug and/or if the amount of protein absorbed or deposited on the interior of a stabilizing catheter is reduced, when either “site loss” or “protein absorption/deposition” are compared to the amount of protein lost, absorbed or deposited using the same protein drug and a prior art, non-stabilizing catheter.

A protein drug formulation, such as an insulin formulation, is “stabilized”, “maintained”, “preserved” or the like, in embodiments of the present invention if flow-impeding or flow-blocking deposits/occlusions do not form during a predetermined period set for infusion of insulin.

A protein drug or protein drug formulation, such as insulin, is “destabilized” as it traverses through a non-stabilizing catheter when the biologically/pharmacologically active form of the protein is not maintained or preserved for delivery to a desired site within the body. Thus, in embodiments of the invention, the processes of “protein destabilization” and “protein formulation destabilization” results in changes to the active form of the protein to be delivered. Further, the process of “protein formulation destabilization” also results in changes in the composition of the protein formulation, as well as changes to the protein itself.

The phase “biologically/pharmacologicaly active form of protein” includes complex protein drugs, such as insulin, that may reversibly exist in multiple forms, such as monomers, dimers, tetramers, hexamers, or the like. For insulin, the form of the protein drug depends on variables such as concentration, pH, as well as the type and amounts of excipients contained in a particular formulation. Thus, while resident in a catheter, for example, insulin may exist largely as a hexamer, depending on protein concentration and other factors. However, upon delivery to a site within the body, insulin may exist in other forms such as a monomer or dimer, or the like. In embodiments of the invention, the stabilizing catheters maintain or preserve a biologically/pharmacologically relevant form of the protein, so that the biological/pharmacological effects of the protein drug upon delivery can be observed.

The term “occlusion” or the like describes an protein-containing blockage, which also may include any excipient of a particular protein formulation, such as a buffer component, an excipient or other adventitious small molecules, located along or within the lumen wall of a delivery catheter which substantially impedes or blocks fluid flow during a period set for insulin delivery. These protein deposits may stick to the interior walls/surfaces or the delivery catheter and/or may fall off and be delivered to a site within the body.

The term “deposit” refers to deposition or absorption of proteins on the walls/surfaces of a delivery catheter.

An “impeded or blocked fluid flow”, or the like, is one that necessitates rinsing of a catheter or changing a catheter to insure infusion of a desired amount of a protein drug is delivered at a desired rate of delivery.

A “high concentration insulin formulation” is any insulin formulation containing 100 U/ml or greater of any form of insulin.

The term “small molecule” refers to any molecule with a molecular size less than 500 g/mole, including neutral molecules, encompassing polar and nonpolar molecules, and charged or ionic molecules, encompassing positive and negative ions and zwitterions. As used in the art, the term “molecular weight” generally excludes hydration and counter ions.

The terms “monomeric human insulin analog”, “monomeric insulin analog” and “human insulin analog” are well-known in the art. These terms generally refer to fast-acting insulin analogs, typically a human insulin analog where Pro at position B²⁸ is substituted with Asp, Lys, Leu, Val or Ala and where the lysine at B²⁹ is substituted with Pro or where Pro at position B²⁸ is replaced with aspartic acid. However, all known and future developed insulins, including insulin analogs, are included in embodiments of the invention.

The term “Tris” or “Tris buffer” refers to 2-amino-2hydroxymethyl-1,3-propanediol or tris(hydroxymethyl)aminomethane, and to any pharmaceutically acceptable salt thereof The free base and the hydrochloride form are two common forms of Tris. Tris is also known in the art as tris(hydroxylmethyl)aminomethane.

The term “phenol” generally refers to art accepted phenolic preservatives, such as phenol, chlorocresol and m-cresol. However, any phenolic derivative is included in the present invention.

The terms “catheter” or “delivery catheter”, or the like, are used herein to refer to a tubing, including one or more layers, where the tubing serves as an protein drug and protein drug formulation conduit from a reservoir to a desired site for drug delivery within the body of a patient or a user. The term “stabilizing catheter” includes the definition given here for “catheter”, or the like, but also includes at least one layer of a stabilizing material as disclosed below.

II. Characterization of Embodiments of the Present Invention

As shown in the drawings for purposes of illustration, embodiments of the present invention includes improved catheters for use with protein infusion device therapy with either an external infusion device or an internal, implantable infusion device. The stabilizing catheter embodiments of the present invention are particularly suitable for insulin infusion therapy.

Accordingly, these embodiments provide improved methods and devices for maintaining the integrity of insulin and insulin formulations by inhibiting or reducing physical and/or chemical changes of insulin and/or of an insulin formulation that may occur as the protein or the protein-containing formulation traverses along the path of a delivery catheter during infusion of insulin to a site within the body. However, embodiments of the stabilizing catheter of the invention are generally suitable for use for delivery of any protein drug to a site within the body.

One problem of protein destabilization occurs as a protein, such as insulin, flows through a delivery catheter, either an external or internally implanted catheter, and contacts surfaces that have a much lower surface tension than water, i.e., surfaces that are more hydrophobic than the exterior of the protein. As a consequence of these interactions, the proteins is destabilized as it becomes at least partially denatured or unfolded. The destabilized or denatured form of the protein, which now may have exposed hydrophobic amino acids on its surface, can then stick to the hydrophobic surface forming protein deposits and protein aggregates. Further, these aggregate forms of denatured proteins are generally not biologically/pharmacologically active when delivered to a site within the body.

Another problem that is observed as a protein formulation traverses through a delivery catheter is that of protein formulation destabilization which is particularly exacerbated for implanted catheters as these are placed in an in-vivo, aqueous environment which is generally different from that of the external, air environment. In particular, the concentration of dissolved gases, such as CO₂ and O₂, is different in-vivo as compared to ambient air. For example, in-vivo O₂ levels are approximately 4-5%, whereas ambient air contains approximately 21% O₂. Conversely, the levels of CO₂ are generally greater in-vivo than in ambient air. Further, in the aqueous environment provided in-vivo, other non-gaseous, small molecules abound which are not present outside the body. Thus, although embodiments of the invention apply to both external and internal infusion systems, a stabilizing catheter embodiment that provide a barrier to diffusion of small molecules may be particularly useful as applied to internally implanted infusion systems.

1) Discovery of the Problems Solved By Embodiments of the Present Invention

Particular embodiments of the invention are based on the unexpected discovery that “site loss” of insulin occurs during protein infusion therapy, particularly for the delivery of monomeric insulins, such as human analogues of insulin, i.e., LISPRO or the like. Site loss refers to an apparent, yet unexplained, hyperglycemic event following the delivery of insulin from an external infusion device to a subcutaneous delivery site. Further, site loss is generally accompanied by inflammation at, near or surrounding the subcutaneous delivery site.

Without being held to a particular theory, embodiments of the present invention are based on the notion that the phenomenon of site loss is due to the denaturation, unfolding or degrading of insulin as it contacts the surfaces of a delivery catheter. In this scenario, the denatured insulin is in a form that is not biologically/pharmacologically active, and thus, is essentially not bioavailable. As a consequence, even after a bolus of insulin is delivered to a patient, there is no apparent insulin effect and hyperglycemia is observed. Further, the inflammation surrounding the subcutaneous delivery site results from an undesired immunological response to denatured or aggregated, i.e., non-native, forms of insulin.

The phenomenon of site loss has been observed for external insulin infusion, but can apply to implantable infusion insulin infusion devices. Moreover, site loss may be more prone to occur when using monomeric insulins, such as LISPRO or the like. As described below, monomeric insulins require phenol and/or zinc to increase their stability by forming the more stable, hexamer, form of insulin.

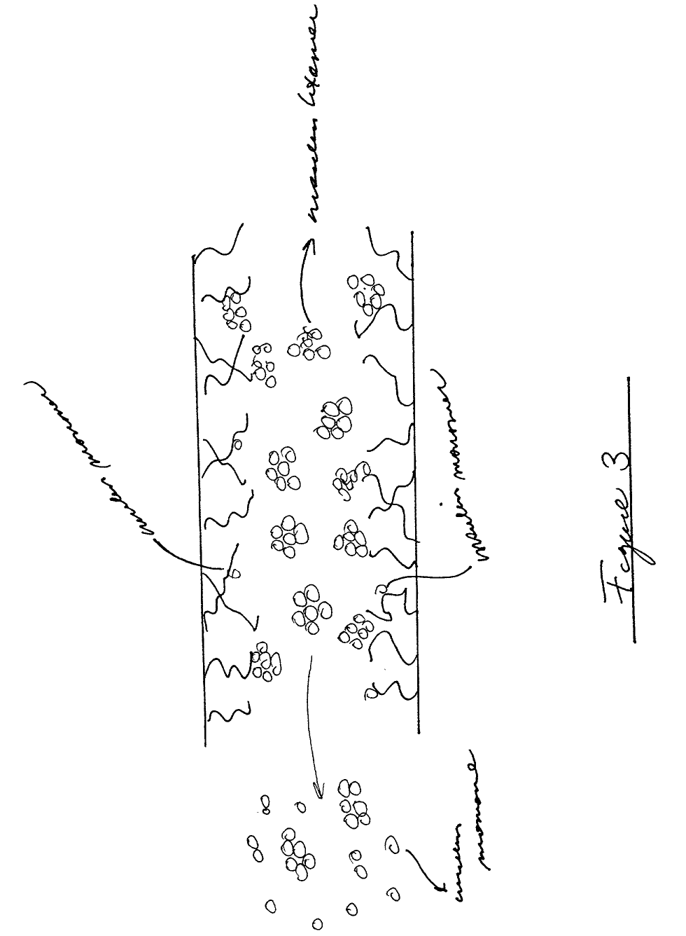

It has been observed that when a delivery catheter is coated with a hydrophilic substance, preferably any polyeythylene glycol-containing polymer, site loss of insulin is diminished, as illustrated in FIG. 3. Hydrophilicity is a character of materials exhibiting an affinity for water. The surface chemistry of these materials thus allows for wetting, i.e., forming a water film on the surface, increased surface tension and the ability to form hydrogen bonds with water and other molecules that have an affinity for water, such as proteins. The converse of a hydrophilic material is a hydrophobic material. These materials have an opposite response to water as compared with hydrophilic materials. Thus, hydrophobic materials have little or no tendency to absorb water, possess low surface tension values and generally lack chemical groups that can hydrogen bond with water. As a consequence, when a protein drug interacts with a hydrophobic surface, the protein tends to denature, or unfold, because the hydrophobic amino acids of the protein which are generally contained in the interior of the protein structure are driven outward in response to contacting the hydrophobic surface.

Hydrophilic substances suitable for use in the present invention should also possess mobility. The characteristic of mobility of a hydrophilic polymer coating may depend on factors such as the chain length of the polymer and the degree of rotational freedom around the atoms of each repeating unit of the polymer. Further, the characteristic of mobility may not permit strong hydrogen bonding, ionic, ion-dipole, dipole-dipole, van der waals, or the like, interactions to accompany contact interactions of the protein drug with the polymer coated surface, so that the protein drug does not stick to the surface.

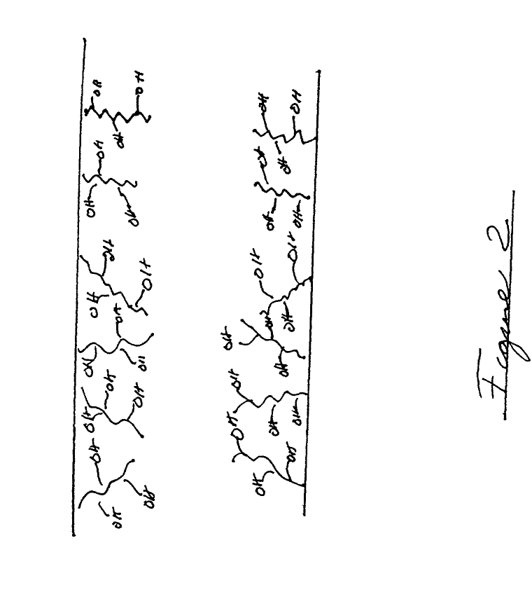

Thus, in combination, these two properties maintain the 3-dimensional structure of a protein drug and/or do not substantially allow proteins to stick to the surface following contact, as illustrated in the stabilizing catheter design shown in FIG. 2. As a consequence, the protein drug is delivered to a site within the body in a form that produces the desired biological/pharmacological effect and the phenomenon of “site loss” is reduced or eliminated.

Other embodiments of the invention are based on unexpected discovery that catheter flow-impeding deposits/occlusions occur when an insulin formulation is delivered via an implanted catheter, especially when using an analogue insulin formulation, such as a LISPRO insulin formulation. This result is unexpected because analogue insulin formulations, which are largely monomeric and fast-acting, are generally much more stable than nonanalogue insulin formulations, such as semi-synthetic insulins and recombinant insulins.

Mass spectral analysis of these flow-impeding deposits/occlusions showed that the major component was insulin and Tris in a 1/10 ratio. These results also were particularly unexpected since both Tris and insulin are very soluble at the concentrations used in the formulations.

Without being held to a particular theory, a hypothesis is given herein to explain these unexpected results. It is first hypothesized that a reaction between CO₂, Tris and insulin is involved in the formation of these deposits/occlusions/deposits for at least two reasons. The first reason is that no deposits/occlusions occur when pumping these formulations in-vitro. The second reason is pH-induced precipitation can be an artifact of pumping insulin through catheters that have a high permeability to CO₂.

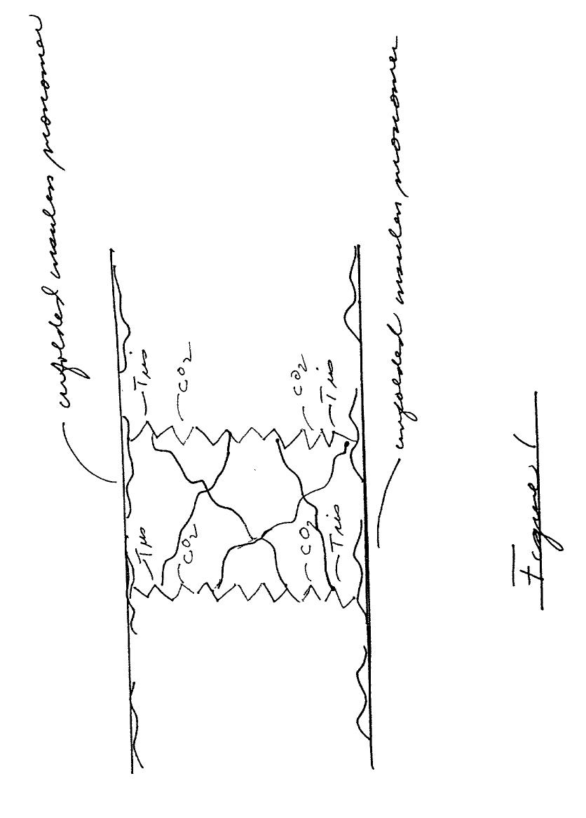

Thus, embodiments of the invention are based on an understanding that CO₂ dissolved in the insulin formulation consumes Tris by reacting with it to form a carbamide, especially at higher concentrations of CO₂. This chemical process results in a reduction in the buffering capacity of the Tris and a concomitant reduction in the pH of the formulation. The resultant drop in the pH may destabilize insulin leading to possible denaturation (unfolding of the native protein structure, at least partially) and/or degradation (at least partially) of the protein. The destabilized insulin then may form initial deposits on the wall of the catheter. Or absorbed or deposited insulin may already have coated the walls of a delivery catheter, according to the processes described above. Regardless of how these insulin deposits are initially are formed, these deposits subsequently can lead to the formation of a crosslinked matrix if CO₂ is allowed to influx the catheter. The end result of this scenario is the formation of a physio/chemical occlusion that includes insulin, Tris and CO₂ in a approximate 1:10:5 ratio as illustrated in FIG. 1. The hypothesis that an influx of CO₂ led to the formation of these deposits/occlusions was tested and the results are given in Example 5.

Further, the formation of a carbamide is not limited to insulin. The formation of destabilizing carbamides can form with any protein containing an accessible free amine group, which is most known proteins. Thus, the discovered phenomenon of protein deposits leading to flow-impeding occlusions can occur for the delivery of any protein drug to a site within the body, if CO₂ is allowed to influx a delivery catheter particularly when using an amine containing buffer such as Tris.

Moreover, it is further hypothesized that loss of excipients, such as phenol and/or zinc, from a delivery catheter are involved in the destabilizing events that lead to the formation of deposits/occlusions. This hypothesis is based on experimentation that has shown that phenol can be lost from a delivery catheter over time. The results are given in Example 1. These results add to the above hypothesis in that phenol and its derivatives help to stablize insulin, especially insulin analogues. For instance, it has been found that bacteriostatic substances, such as phenol and its derivatives, have a dual functionality in that these substances additionally stabilize insulin by inducing one or more protein structural transformations.

Embodiments of the present invention are based on the fact that the presence of phenol and zinc stabilize insulin analogues, such as LISPRO. These excipients act by promoting the formation of the hexamer form of insulin which is generally more stable to denaturation and/or degradation than monomeric insulin. See Ciszak, E. et al., Structure (1995) Vol. 3, No. 6, p. 615. Embodiments of the present invention are also based on the fact that phenol stabilizes certain alpha-helical portions of the insulin monomer. See Birnbaum, D. T., et al., Pharamceutical Research (1997) p.25. In embodiments of the present invention, it is theorized that this structural stabilization may play a role in stabilizing monomeric insulins, such as LISPRO.

It is further theorized that monomeric insulins, such as LISPRO are more prone to destabilization from denaturation/degradation if the phenolic concentrations are not maintained properly during insulin delivery, especially as an insulin formulation traverses through a deliver catheter. Thus, it is theorized that the increased stabilization of insulin by phenol, given that this excipient may stabilze both the monomeric and hexameric forms of insulin, substantially reduces denaturation/degradation, which in turn substantially decreases the initial formation of deposits along the walls of the catheter that lead to the formation of flow-impeding deposits/occlusions. Additionally, the phenol-induced alpha-helical transformation also has been found to reduce deamidation of the insulin molecule, and thus, it is theorized that phenol reduces the chemical degradation of insulin that leads to subsequent precipitate formation and flow-impeding deposits/occlusions.

Further, chemical degradation and polymerization of insulin also leads to precipitation. These two chemical reactions are generally induced by changes in pH. Hydrolytic decomposition of insulin generally proceeds as the pH is lower and reflects the increasing dissociation of insulin hexamers into dimers and monomers as a function of decreasing pH. On the other hand, polymerization reactions, due mainily to disulfide interchange reactions and resulting in oligomers and polymers of insulin, are more prevalent as the pH is increased from neutrality. Moreover, insulin generally start to form precipitates at pH lower than 6, given that the isoelectric point of the insulin molecule is approximately 5.4. Thus, it can be concluded that certain chemical entities and chemical environments have been found to have a profound effect on stabilizing, or destabilizing, the native structure of insulin.

Embodiments of the present invention, therefore, are based on the discovery that certain small molecules having a stabilizing effect on an insulin formulation diffuse out from a delivery catheter and formulation-destabilizing small molecules diffuse into a delivery catheter during delivery to a site within the body. These diffusional processes result in changes to the integrity of an insulin formulation as it moves through a delivery catheter. These processes may result in destabilized (denatured/degraded) insulin monomer coating the interior walls of the delivery catheter. A concomitant process is the formation of deposits/occlusions as Tris-CO₂ begins to react with the deposited insulin. The final result of this process is evidenced by deposits/occlusions, which impede or block fluid flow, being formed at one or more points along the lumen of the catheter. A depiction of an occlusion within the lumen of a delivery catheter is shown in FIG. 1.

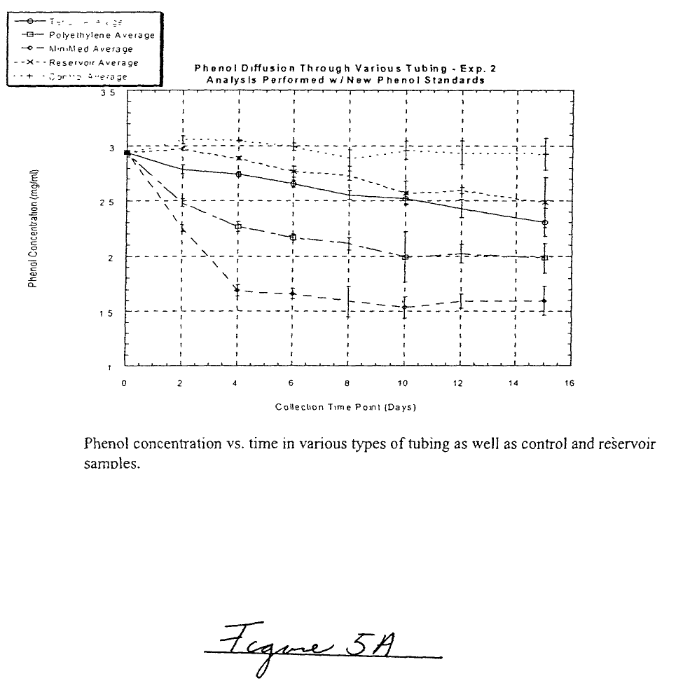

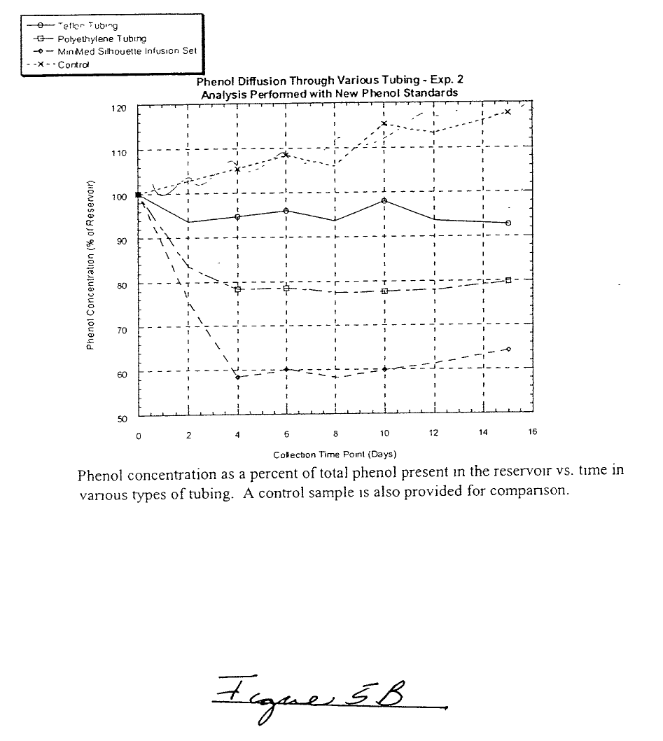

Moreover, particular embodiments of the present invention are based on the discovery that when implanted catheters of the prior art are in use, phenol is lost at a greater rate via the delivery catheter, as compared with residual phenol loss in the implanted insulin reservoir during a given time period. These experimental results are shown in FIG. 5A and FIG. 5B. These results also apply to phenolic derivatives. This loss of the insulin stabilizer, phenol, may be one of the initial causes of deposits/occlusions being formed within, or on, the interior lumen of the catheter during delivery of insulin as insulin may be more prone to denaturation as phenol diffusing through a delivery catheter.

Further, prior art, implanted catheters are generally permeable to carbon dioxide. A result of the flow of carbon dioxide into the delivery catheter may be a change in the pH of the insulin formulation As discussed above, a change in pH can result in destabilization of the native structure of insulin and subsequent precipitation. Evidence of the destabilizing effect of carbon dioxide diffusion into a delivery catheter is presented in Example 5.

Given these destabilizing processes, such as protein denaturation and diffusion of small molecules which occur with the use of current, state of the art, catheters, embodiments of the present invention are directed to improved catheters that includes stabilizing materials which maintain a biologically/pharmacologically active form of a protein drug and/or which impede diffusion of small molecules, thus maintaining or preserving the integrity of a particular protein drug protein and drug formulation.

2) Stabilizing Catheter Embodiments of the Invention

Embodiments of the present invention are directed to providing stabilizing catheters that substantially maintain a biologically/pharmacologically active form of the protein drug to be delivered to a site within the body as it flows through the stabilizing catheter. Embodiments of the invention accomplish this end by various means. Particular stabilizing catheter embodiments in accordance with the invention provide appropriate protein-compatible surfaces along the interior of stabilizing catheters, such that interactions of the protein drug with these surfaces do not denature the protein. Still other embodiments of the invention provide stabilizing catheters that substantially reduce the diffusion of small molecules into and out from a delivery catheter during a period set for protein drug delivery, still other embodiments of the invention provide both a protein compatible surface and a barrier to diffusion of small molecules. These latter embodiments also provide stabilization to insulin itself which can be destabilized by changes in its environmental milieu.

Catheter embodiments of the present invention maintain a biologically/pharmacologically active form of a protein and/or impede diffusion of small molecules both into and out from the stabilizing catheter. In effect, embodiments of the stabilizing catheter maintain or preserve the active form of particular protein drugs, as well as maintain or preserve particular protein drug formulations. That is, catheter embodiments of the invention maintain a protein drug and protein drug formulation so that these do not substantially change as the protein drug or the protein formulation traverses through a delivery catheter. Thus, the protein drug or protein drug formulation is maintained, as compared to the protein drug or protein drug formulation found in the reservoir, during a given time period set for protein drug delivery. In particular embodiments, the protein drug and protein drug formulation delivered include insulin.

Embodiments of the present invention are related to Ser. No. 09/042,138, filed Mar. 13, 1998, which is a continuation application of U.S. patent application Ser. No. 08/742,377, filed Nov. 1, 1996; this application is also related to Ser. No. 09/324,783, filed Jun. 3, 1999. The contents of each of these related applications have been incorporated herein by reference in their entireties. In these related applications, the preferred method of attaching hydrophilic polymers to a surface is through covalent bonding of the polymer to a treated surface. However, embodiments of the present invention are not limited to covalently linking polymers to the interior surfaces of a delivery catheter. Embodiments of the invention include polymer coatings that are affixed or adhered in any manner. In particular embodiments where a stabilizing catheter is used with an external infusion device, the stabilizing coating that lines the interior of the catheter could be physically absorbed or adhered to the surface because of the short term usage of these external devices. Thus, in these embodiments, the coating would not have to withstand long-term use. However, for implantable infusion devices, covalent attachment of a stabilizing polymer is preferred.

The invention can be applied to a wide range delivery catheters found in both reusable and non-reusable pumps, as well as to both implantable and externally worn pumps. For example, the invention is applicable to an externally worn, gas powered infusion device as described in U.S. Pat. No. 5,785,688; an implantable constant-flow medication infusion pump as described in U.S. patent application Ser. No. 08/871,830, and the pumps described in U.S. patent application Ser. No. 09/253,382 and Ser. No. 09/253,383 the disclosures of which are incorporated herein in their entireties by reference, as well as other medical devices that employ flexible displaceable membranes.

The invention can further be applied to a variety of delivery catheter surfaces including both metallic and non-metallic surfaces to reduce the surface contact angle so as to yield hydrophilic characteristics. The adsorption and subsequent denaturation of the protein-based medication on a surface is functionally related to its surface free energy. Accordingly, embodiments of the present invention relate to a stabilizing catheters where one or more internal surfaces are coated to achieve a significant reduction in surface free energy such that the ability of such surfaces to destabilize proteins such as insulin is reduced. A variety of insulin proteins that are stabilized by such surface treatments are well-known in the art, including human and porcine or bovine insulin as well as to fast acting analogs of insulin (typically human insulin), which include: human insulin, wherein Pro at position B28 is substituted with Asp, Lys, Leu, Val, or Ala, and wherein position B29 is Lys or is substituted with Pro; AlaB26-human insulin, des(B28-B30) human insulin; and des(B27) human insulin. Illustrative insulin proteins are disclosed in U.S. Pat. No. 5,514,646, WO 99/64598, WO 99/6459A2 and WO 96/10417A1.

Delivery catheters having surfaces comprised of both metallic and non-metallic materials, and components of such medical devices which are comprised of both metallic and non-metallic materials, are beneficially prepared according to embodiments of the present invention. The metallic surfaces can be comprised of, for example, titanium. The non-metallic surfaces can be comprised, for example, of a polymeric material, for example a rubber such as bromobutyl rubber or chlorobutyl rubber, a polyurethane, a polyethylene, a polypropylene, a polyvinylchloride, or other similar polymeric materials. The medical device components can be made of a polymeric material, such as those listed above, or can be formed from a polymer laminate (e.g., two or more layers of different polymeric materials) or a metallized polymeric material, in which case the polymeric material has a nonmetallized surface which has a surface treatment according to the invention.

The surface treatment according to the invention can be, for example, a coating formed from a polymeric material. Specific polymeric materials useful to provide a surface treatment according to the invention include, without limitation, materials such as hydrophilic polyurethanes, polyureas, acrylics, as well as other hydrophilic components. Particular materials include polyethylene glycols, polyethylene/polypropylene glycol copolymers and other poloxamers. These coatings preferably are covalently bonded to the surface which is being treated.

One particular method for forming the coating includes the steps of adsorbing the polymeric material to the surface, and then covalently attaching the polymeric material to the surface by exposure to UV radiation, RF energy, heat, X-ray radiation, gamma radiation, electron beams, or the like. If needed, the foregoing application and curing steps are carried out at least twice, more particularly at least three times, in order to avoid bubble formation and provide uniform surface coverage.

Another particular method includes the step of covalently attaching a linker molecule to the surface. Linker molecules that are useful in this embodiment of the inventive method include, without limitation, silanes of the formula SiX3—R, wherein X is a methyl group or a halogen atom such as chlorine and R is a functional group which can be a coating material as described herein or a group which is reactive with a coating material. Particular silane-terminated compounds include vinyl silanes, silane-terminated acrylics, silane-terminated polyethylene glycols (PEGs), silane-terminated isocyanates and silane-terminated alcohols. The silanes can be reacted with the surface by various means known to those skilled in the art. For example, dichloro methyl vinyl silane can be reacted with the surface in aqueous ethanol. The linker molecule strongly binds to the surface via —O—Si bonds or directly with the silicon atom. The vinyl group of the silane can then be reacted with polymeric materials as described herein using appropriate conventional chemistries. For example, a methacrylate-terminated PEG can be reacted with the vinyl group of the silane, resulting in a PEG that is covalently bonded to the surface of the medication device.

In accordance with a preferred surface treatment and method, a hydrophilic polymer, including hydrophilic surfactants, is applied to the selected surface of the medical device to significantly reduce adsorption of a protein-based medication such as insulin. Several hydrophilic surfactants are available for this purpose, including Genapol™, a block ethylene/propylene copolymer having a molecular weight of about 1800 Daltons, available from Hoechst Celanese Co. of Somerville, N.J. Other hydrophilic surfactants include Tween, a polyoxyethylene sorbitan available from Sigma Biochemicals of St. Louis, Mo. and Brij, a polyoxyethylene ether also available from Sigma Biochemicals of St. Louis, Mo.

Due to the highly heterogeneous structural and chemical characteristic of different proteins, those skilled in the art assess the compatibility between a specific protein such as insulin and the hydrophilic surfactant that is applied to the selected surface of the medical device to reduce adsorption of a protein-based medication (e.g. PEG). In this context, artisans understand that proteins are amphiphilic substances which have very different characteristics that influence their interaction with other molecules such as hydrophilic polymers known in the art. Specifically, different sequences of the various amino acids in the primary sequence of a polypeptide condition the formation of the hydrophilic and hydrophobic regions within the protein and the repulsive and attractive forces between these regions are balanced to form the complex three dimensional structure of the protein's native state. As it is not possible to predict exactly how a specific protein and hydrophilic polymer will interact, each hydrophilic surfactants that could be used to coat surfaces of medical devices is assessed to determine whether it has a structure that promotes the maintenance of that protein's unique native state (i.e. the non-denatured state). As disclosed herein, hydrophilic polymers, including surfactants, which include a polyethylene glycol (PEG) moiety as their hydrophilic segment are highly compatible with protein drugs, particularly with insulin, and promote the maintenance of this specific protein's native state. Most importantly, these hydrophilic polymers function to preserve the complex three dimensional structure of insulin even when they are covalently attached to a substrate known to denature this protein.

Covalent modifications to hydrophilic polymers may involve the generation reactive polymer sites which then covalently attach the polymer to a surface, a process which alters the complex 3D architecture of the polymer. Because this process alters the architecture of polymers, such modifications can correspondingly effect their protein stabilizing properties. Consequently, it is not possible to predict exactly how such covalent modifications will effect each hydrophilic polymer's ability to promote the stability of a specific protein and whether the stabilizing property of a given polymer will be compromised by such modifications. This unpredictability is illustrated for example, by reports that small changes to the side chain structures of various polymers (or even alterations to their molecular weights) impact their ability to stabilize proteins (see, e.g., Thurow et al., Diabetologia (1984) 27: 212-218).

The observation that polymers such as Genapol PF-10™ retain desirable properties even after being chemically modified and covalently affixed to a matrix is surprising in view of reports that teach that even slight alterations to these polymers can dramatically effect their ability to stabilize proteins. Moreover, it is believed that the active principle of polypropylene glycol polymers involves how the alternating arrangement of hydrophobic lateral methyl groups and hydrophilic oxygen bridges in the polymer contact a hydrophobic interface. Consequently it is surprising that covalent modifications to such polymers which have the potential to disturb this arrangement do not in fact compromise the polymer's ability to promote the stability of a protein. As disclosed herein however, certain polymers such as the polypropylene glycol/polyethylene glycol polymer described below can be covalently modified in this manner and still generate an improved surface, i.e. one that defines a surface contact angle less than about 45 degrees and exhibits an insulin adsorption profile of less than about 1.0 microgram per square centimeter.

The hydrophilic polymers can be attached to a selected surface by any one of a wide variety of methods known in the art. Typically, the polymer is covalently attached to the surface by a method selected from the group consisting of polymeric attachment, RF-plasma attachment, grafting, or silane-based primer attachment. The invention disclosed herein has advantages over previously described coating methods because the polypropylene glycol/polyethylene glycol polymers can be securely affixed to a surface via covalent attachment. In addition, as noted above, a significant and surprising finding is that these polymers continue to inhibit the denaturation of insulin even when their chemical structure is modified as part of the covalent attachment process.

Protein adsorption is significantly reduced as a result of the inventive surface treatment, typically to about 1.0 microgram or less per square centimeter of the treated surface, more specifically when measured with insulin. For example, insulin adsorption after Genapol™ surface treatment is less than 0.1 microgram per square cm of the surface, as compared to an adsorption of about 1.5 microgram per square cm for the uncoated surface. Similar surface treatments using other hydrophilic surfactants such as those identified above yield results of similar magnitude, although PEG containing polymers are believed to provide the best reduction in insulin adsorption.

A further alternative coating method in accordance with the invention utilizes a hydrophilic polyurethane, such as that marketed by Thermedics, Inc. of Woburn, Mass., under the name Biomer. In this method, Biomer is prepared in an approximate 7.0% solution with tetrahydrofuran (THF) and the surface to be coated is dipped therein. The dip coated surface is subsequently dried for about six hours at about 45 degrees Celsius. Subsequent hydration as by exposure to water for about one hour results in a surface contact angle and insulin adsorption profile that is too low to measure, i.e., less than about 0.04 micrograms per square centimeter.

A hydrophilic surface coating can also be prepared by the use of bovine serum albumin (BSA) dissolved in a phosphate buffered saline (PBS) solution with a concentration of about 5 milligrams per milliliter. The medication device surface to be coated is dipped into this solution and allowed to dry. After drying, the coated surface is dipped a second time into the BSA solution and then immediately dipped into a solution of glutaraldehyde in deionized water with a concentration of about 2.5% which functions to cross link the protein both to the surface and also to itself. After drying for about two hours, at about 37 degrees Celsius, the resultant surface contact angle is about 30 degrees, and it is believed that a comparable reduction in insulin adsorption will result.

There are several ways to covalently attach a hydrophilic coating to the surface of the medication device. These include radiation, electron beam and photo induced grafting, polymerization chemical grafting and plasma deposition of polymers. In general, these methods involve an energy source and a monomer of the desired hydrophilic polymer. For example, acrylonitrile can be grafted onto a surface by irradiation of acrylonitrile vapor in contact with the surface. The resulting polymer, polyacrylonitrile (PAN) has excellent hydrophilic properties with very minimal protein interaction with the surface. A wide variety of polymers can be produced in this manner, the only requirement being that the monomer be available in reasonable purity with enough vapor pressure to be reactive in the deposition system.

Accordingly, the present invention provides a treated surface exhibiting significant hydrophilic properties, with a reduced surface contact angle, preferably of less than about 45 degrees, and more preferably less than about 35 degrees. This treated surface has a low free energy and has provides demonstrated protein compatibility.

Other embodiments substantially preserve or maintain a particular insulin formulation including various excipients, as well as chemical environments, such as pH, the integrity of insulin in a particular insulin formulation is concomitantly preserved. In embodiments of the present invention, the stabilization/maintainance of an insulin formulation generally is evidenced by a lack of deposits/occlusions being formed in the stabilizing catheter during a period set for insulin infusion. The stabilizing catheter provides a sufficient barrier to limit interaction between a particular insulin formulation and the in-vivo, chemical environment provided by the body, where the levels of dissolved gases and other small molecules are different from that of ambient air. Thus, embodiments of the present invention preserve an insulin formulation as it moves through the stabilizing catheter to a desired site of delivery in the body.

In particular embodiments, the stabilizing catheter is used with an implantable insulin infusion system for intraperitoneal insulin delivery to a patient or user. However, embodiments of the stabilizing catheter may be used for any drug delivery system, including both internal, implanted infusion devices or external infusion devices.

In embodiments including an implantable infusion device, the implanted stabilizing catheter carries an insulin formulation from the infusion device to an exit tip of the stabilizing catheter positioned at a delivery location within the body. Often insulin is delivered via the portal circulation to simulate the body's natural release of insulin. Alternatively, the insulin is released into other cavities of the body, directly into the blood stream, into subcutaneous tissue, or the like.

One particular concern associated with insulin instability is pump failure caused by the formation of destabilized and/or degraded insulin products. During infusion of insulin, these insulin products can be deposited in the lumen of the delivery catheter resulting in blockages to fluid flow.

One new class of insulin molecules is represented by monomeric insulin analogs. These insulin analogs are known as rapid-acting insulins, as disclosed in Chance, et al., U.S. Pat. No. 5,514,646, and herein incorporated by reference in its entirety. Additionally, monomeric insulin analogs are absorbed in the body much faster than is insulin, and consequently, are especially well-suited for postprandial control of blood glucose levels. These insulin analogs also are especially well-suited for administration by infusion for both prandial and basal control of blood glucose because of their rapid absorption from the site of administration. Generally, these insulin analogues are more stable than non-analogue insulin.

In this regard, formulations of U400/ml insulin analogs, such as U400/ml insulin LISPRO (B28 Lysine, B29 Proline), have been developed to be used with implantable pump therapy. LISPRO formulations comprising 400 U/ml of insulin are preferred when using an implantable pump because of their improved stability and because the high concentration of the formulation permits the insulin pump reservoir to be more compact and/or require less frequent refilling.

In general, embodiments of the implantable insulin infusion systems include a reservoir, a negative pressure chamber, a motor, electronics, a power supply and a stabilizing catheter. However, alternative embodiments my utilize a constant pressure device, such as those disclosed in U.S. Pat. No. 5,957,890, which is herein incorporated by reference in its entirety.

The reservoir is typically refillable and is filled with insulin for delivery/infusion into the body. In certain embodiments of implantable infusion systems, the negative pressure chamber is a safety feature designed to apply negative pressure to the reservoir, which, in the absence of other forces, draws the insulin into the reservoir and prevents it from leaving the reservoir. When the motor is actuated, the pumping force of the motor must overcome the negative pressure caused by the negative pressure chamber in order to pull insulin out of the reservoir and pump it into the stabilizing catheter. The motor is activated by the electronics, which are typically programmable to control the rate insulin is infused into the body. The power supply provides power to operate the electronics and actuate the motor. In preferred embodiments, the insulin infusion systems are of the type described in U.S. Pat. Nos. 4,373,527; 4,525,165; 4,573,994; 5,957,890; 5,167,633; 5,176,644; 5,514,103; 5,527,307; 5,569,186; and 5,665,065; or the like, which are herein incorporated by reference in their entireties.

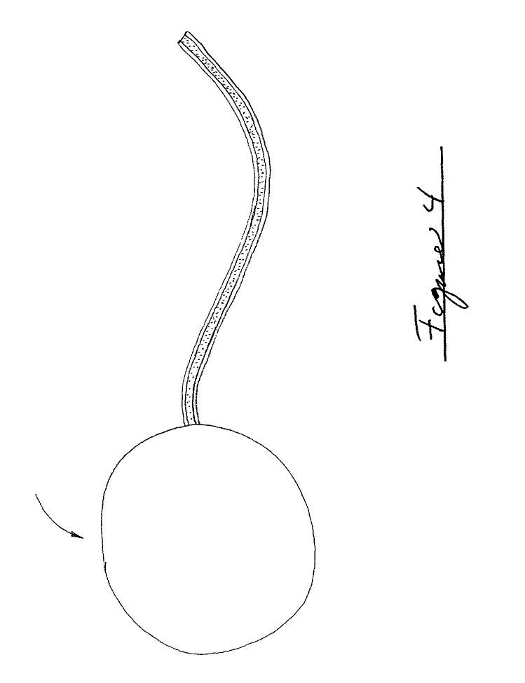

In preferred embodiments, the stabilizing catheter includes a tubing with a connector coupled to one end and an exit tip on the other end, as exemplified in FIG. 4. The stabilizing catheter may have various geometries and connectors, such as described in U.S. Pat. Nos. 4,531,937; 4,826,480; 4,947,845; 5,460,618; 5,505,713; 5,538,511; 5,788,678; 5,807,315; and 5,868,720; or the like, which are herein incorporated by reference in their entireties. In preferred embodiments, the stabilizing catheter has a minimum wall thickness of about 0.100 in. (about 0.254 cm), a length of about 10.0 in to about 15.0 in (about 20.54 cm to about 38.10 cm), and a minimum inner diameter of about 0.05 in (about 0.127 cm). The stabilizing catheter wall thickness may be increased or decreased and/or the inner diameter increased or decreased depending on the diffusional stabilizing materials selected for use in particular embodiments of the stabilizing catheter. Moreover, the precise configuration of an embodiment of the stabilizing catheter may affect the overall wall thickness, as well as the inner diameter.

In preferred embodiments, a physician fills or refills the reservoir with insulin using a syringe or other filling device. The reservoir may hold enough insulin for several days, weeks or even months of treatment depending on the insulin concentration and the patient's daily insulin requirement. Insulin formulations for traditionally, self-administered syringe injections typically have insulin concentrations of U40 or U100 (40 or 100 units of insulin per milliliter of solution), which are dilute enough for patients to accurately measure the dosage while manually filling a syringe.

Since continuous insulin infusion therapy provides very fine dosage resolution by providing a variety of basal infusion rates to the patient where microliters of insulin are infused over time, higher concentration insulin formulations may be used. In preferred embodiments of the invention, the insulin concentration is U400 (400 units of insulin per milliliter of solution), although higher (up to about U1000) or lower (down to about U10) concentrations can be used in embodiments of the invention. Moreover, insulin formulations with increased insulin concentrations are desirable for implantable embodiments of the infusion systems because as insulin concentrations increase, the fluid volume required per dose decreases, and concomitantly, the frequency that a patient must visit a physician to refill the reservoir decreases.

The problem of insulin formulation destabilization includes insulin precipitation at one or more points along, or within, the internal walls of the catheter or on other infusion device control surfaces that lead to deposits/occlusions and cessation of delivery. Correction of the problems may require a large number of infusion device rinsing procedures, catheter replacements, and reduced time intervals between reservoir refills, to reduce the reservoir shelf-life of the insulin.

In preferred embodiments of the present invention, the stabilizing catheter wall includes a layer of one or more materials with low CO₂ diffusional properties, as well as, increased barrier properties to phenolic moleucles. In particular embodiments, the stabilizing catheter is made of Teflon, (polytetrafluoroethane), which has inherently low CO₂ diffusional properties, as well as providing reasonable stabilizing properties to phenolic molecules. A parameter for determining the acceptability of a particular embodiment of the stabilizing catheter in terms of loss of phenol would be less than about 10%, preferably less than about 5%, phenol loss at an insulin infusion rate of about 20 U/day. Additionally, preferred embodiments of the stabilizing catheter will decrease the diffusion of CO₂ into the stabilizing catheter up to about 1000 fold, as well as decrease the diffusion of phenol out from the catheter up to about 100 fold, as compared to prior art, silicone catheters.

In other particular embodiments, the stabilizing catheter is at least partially made of hydrophilic glass, Saran (PVOC), polysulfone, or the like. Additionally, a thin film metal or braided metal material may be used as a stabilizing layer of the stabilizing catheter. A preferred wall thickness for Teflon and Saran is about 0.002 in to about 0.02 in (about 50 to 500 microns). For a stabilizing catheter including a glass fiber layer, wall thickness is immaterial.

In alternative embodiments, the stabilizing catheter is made of multiple layers, one or more layers being Teflon, hydrophilic glass, Saran, polysulfone, or the like, and one or more other layers includes one or more biocompatible, flexible materials. In these embodiments of the invention, an outer layer may either substantially encase an inner stabilizing layer or an outer layer may be applied only partially along the stabilizing layer. In particular embodiments, the outer layer of the stabilizing catheter is a polyurethane, a polyethylene, a silicone, or the like, and the inner layer of the stabilizing catheter includes stabilizing materials as disclosed herein.

In preferred embodiments, the stabilizing catheter wall material has a CO₂ diffusion rate of less than about 3,000 mm/m^{2·24}hr·bar. In alternative embodiments, the stabilizing catheter wall material has a CO₂ diffusion rate of less than about 5,000 mm/m^{2·24}hr·bar.

In preferred embodiments of the invention, insulin analog formulations such as LISPRO (B28 Lysine, B29 Proline human insulin, Eli Lilly), Aspart (Novo Nordisk), or the like, are used. Experimental results have shown that the insulin analog formulations have improved stability when higher concentrations of phenolic preservatives are included in a particular formulation. Thus, when using insulin analog formulations, phenolic agents are generally added for increased stabilization of the insulin molecule. In alternative embodiments, however, other insulin formulations, which utilize other forms of insulin, may be used in the present invention.

An example of a stable formulation of U400 LISPRO for use in the present invention is as follows:

| | | |
|---|---|---|
| a. Insulin | 400 | U/ml (about 15 mg/ml) |
| b. Glycerin | 16 | mg/ml |
| c. Phenol | 0.9 | mg/ml |
| d. m-cresol | 2.2 | mg/ml |
| e. Tris buffer | 2.0-6.0 | mg/ml |

Although the above insulin formulation is preferred, any insulin formulation can be utilized with the stabilizing catheter embodiments of the present invention. However, some testing may be required to ascertain whether a particular insulin formulation is compatible with the particular stabilizing materials chosen for use in the stabilizing catheter.

Given the possibility for incompatibility between a particular embodiment of the stabilizing catheter and a particular insulin formulation, the stabilizing catheter preferably includes a layer of insulin compatible materials, preferably a hydrophilic layer or coating, such as a coating including PEG (polyethylene glycol) or a coating including polyurethane, or the like. Other methods of providing an innermost coating or layer that is compatible with a particular insulin formulation is disclosed in U.S. Ser. No. 09/042,138 where the surfactants, such as Genapol and Tween, are used to coat the interior of a delivery catheter. Other surfactants, such as Triton series and Brij series of surfactants are suitable for use in embodiments of the invention as an insulin compatible innermost layer or coating. Further, insulin formulations, which include appropriate excipients, are to be selected for use with a particular embodiment of the stabilizing catheter of the invention so that the compatibility between an innermost layer of a particular stabilizing catheter and a particular insulin formulation is increased.

An in-vitro evaluation of the stability of this formulation shows that the formulation is stable for at least 1000 hours when tested in an accelerated vial vibration test (vide supra). However, in-vivo testing in a canine model uncovered an unexpected result. During infusion using an implanted device, deposits/occlusions rapidly developed along the lumen of the catheter. These insulin deposits/occlusions blocked the fluid flow through the catheter. Mass spectral analysis of the blockage showed that the major components of the precipitates consisted of insulin and Tris (tris-hydroxymethyl aminomethane) in an approximate 1/10 ratio. As stated above, since both Tris and insulin are very soluble at the concentrations used in the formulation, spontaneous precipitation of these constituents of the formulation does not provide a viable explanation of the observed result. Since no occlusions occurred while infusing these formulations in-vitro and CO₂-induced pH changes can be an artifact of infusing insulin through certain catheters, it appears that, at least, CO₂ diffusion through the catheter wall was in involved the events leading to the formation of the deposits/occlusions.

Further, a polyurethane catheter was used in the in-vivo canine testing. Polyurethane allows a relatively high rate of CO₂ exchange and allows phenol exchange within the body as shown in Example 1 below. An experiment was conducted to test whether CO₂ exchange was the cause of precipitation in the catheter from the canine test disclosed above. The results are disclosed in Example 9. During this experiment, precipitation grew on the walls of the catheter and occlusions developed due to insulin/Tris deposits as confirmed by mass spectral analysis.

From the known properties of insulin, as well as from the data presented in the following Examples, a model is presented that explains the rapid formation of occulsions. This model is included to explain the experimental results, but should not be construed to limit the embodiments of the invention in any manner.

In the model, as the insulin formulation moves through a non-stabilizing catheter, the phenol concentration decreases due to phenol diffusion out from the catheter wall into the body. As the phenol concentration decreases the insulin formulation becomes less stable. This event leads to deposits being formed along the walls of the catheter lumen. At, or around, the same time that phenol is exiting the catheter, CO₂ is entering the non-stabilizing catheter from the body. As CO₂ enters the catheter it can react with an excipient, such as Tris buffer, resulting in a decrease in the pH of the insulin formulation. These changes in the insulin formulation dramatically alter the integrity of the insulin formulation as it moves through the delivery catheter. Concomitantly, when the CO₂ concentration is high enough, the insulin and an excipient, Tris for example, form a complex and this complex binds to the deposited insulin. Subsequently, a fluid-impeding or blocking occlusion forms within the catheter, which largely comprises insulin and a buffer component, such as Tris or any amine-containing or carboxylate-containing buffer or excipient. These occlusions/deposits are substantially precluded from formation by the embodiments of the stabilizing catheter of the present invention.

Embodiments of the present invention are further detailed in the following Examples which are offered by way of illustration and are not intended to limit the invention in any manner.

### EXAMPLES

### Example 1

Experiments were performed to analyze the diffusion of phenol out from various types of implantable catheter tubings and external infusion device tubings. A series of controls were set up where buffer, without phenol, was pumped through various tubing materials. The buffer consisted of 1.6 g/l glycine, 0.6 g/l Tris-HCl and 0.001 g/l Genapol PF10, pH 7.4. A series of phenol standards also were prepared with the following phenol concentrations: 0.7, 1.4, 2.1, 2.8 and 3.5 mg/ml. All standards and samples were read for phenol content at 272 nm. The series of standards yielded a linear relationship between the OD (optical density) at 272 nm and phenol concentration (mg/ml) with a correlation coefficient of 0.9998.

FIG. 5A shows the change in phenol content over time for a variety of tubing materials. The buffer containing 2.8 mg/ml phenol was used to assay the change in phenol concentration over time. The tubing materials compared were Teflon, polyethylene and MiniMed external, which is comprised of PE (polyethylene) lined with PVC (polyvinylchloride) A control was also performed where the change in phenol content over time was monitored for a typical, implantable infusion device reservoir.

As illustrated in FIG. 5A, the Teflon tubing essentially maintains the phenol concentration over the 15 day trial, whereas the MiniMed external lost approximately ½ of the phenol content and the polyethylene tubing lost approximately ⅓ of the phenol content over the 15 day period. FIG. 5B, presents the same data as FIG. 5A, except that the phenol loss from the reservoir has been subtracted from the various curves.

### Example 2

An experimental protocol was developed to compare the rate of formation of protein occlusions in-vitro and in-vivo as this may relate to the different chemical environments surrounding a delivery catheter. For this comparative experiment, one of the most stable insulin high concentration insulin formulations was used. A high concentration LISPRO insulin formulation consisting of 400 U/ml LISPRO insulin (approximately 15 mg/ml), 16 mg/ml glycerin, 0.9 mg/ml phenol, 2.2 mg/ml m-cresol in a Tris buffer (2.0 mg/ml) at pH 7.6 was prepared for use in both the in-vitro and the in-vivo stability tests.

The in-vitro evaluation of the stability of this formulation was conducted in a vial vibration test. The vials were made of glass and held 2.0 milliliters of solution. For this test 2.0 ml of the formulation were place in the vials. The vials were vibrated at a rate of 40 hz at 37 deg C. The data from at least a 10 sample run showed that the formulation was stable for at least 1000 hours when tested in the accelerated vial vibration test (AVVT). No precipitaton was observed during this experiment as evidenced visually and by absorbance at 450 nm.

In-vivo testing in a canine model revealed a different result. An infusion system was implanted in a canine. The infusion system included a polyurethane catheter which was also implanted. Following implantation, a blockage resulted which necessitated removal of the catheter in two weeks. Inspection of the catheter revealed that deposits/occlusions had developed along the lumen of the catheter. This precipitate formation resulted in a total blockage of the fluid flow through the catheter. Mass spectral analysis of the blockage showed that the major components of the precipitate consisted of insulin and Tris in an approximate 1/10 ratio.

### Example 3

An in-vivo experiment is performed using the canine model. The experimental protocol is the same as in Example 4, except that a stabilizing catheter can be connected to an infusion system and implanted into the canine. The stabilizing catheter is made of a single layer of Teflon. After two weeks of being implanted in the canine, the stabilizing catheter can be removed and inspected to ascertain whether protein occlusions are formed during the time period.

### Example 4

An in-vivo experiment is performed using the canine model. The experimental protocol is the same as in Example 4, except that a stabilizing catheter is connected to an infusion system and implanted into the canine. The stabilizing catheter is made of an outer layer of silicone and an inner layer of Teflon. After about two weeks of being implanted in the canine, the stabilizing catheter can be removed and inspected to ascertain whether any protein occlusions are formed during the time period.

### Example 5

An experiment was conducted to test whether CO₂ exchange was a cause of precipitation in the catheter from the canine tests given in Example 4. In this experiment, an infusion system was used to infuse insulin at a controlled rate through catheters that were resident in a water bath at pH 7.4 in a bicarbonate buffer. The pH was maintained by bubbling a 5% CO₂ in air mixture through the water bath. During this experiment, deposits grew on the walls of the catheter and occlusions developed. Mass spectral analysis revealed that the occlusions were formed from an insulin/Tris complex.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. A stabilizing catheter for protein drug delivery to a user, the stabilizing catheter comprising:
a tubing including at least one layer, wherein the at least one layer includes one or more materials that reduce diffusion of small molecules through the tubing, such that when the tubing is used for protein drug delivery, the protein drug formulation is maintained as compared with the protein drug formulation delivered via a different tubing including one or more materials that are free of an effect that reduces diffusion of small molecules through the tubing.

2. The stabilizing catheter of claim 1, wherein an insulin formulation is maintained in the tubing to substantially prevent occlusions or deposits from being formed during insulin delivery.

3. The stabilizing catheter of claim 1, wherein an insulin formulation is stabilized by being substantially free of deposits or occlusions comprising insulin and an excipient.

4. The stabilizing catheter of claim 2, wherein the insulin is a high concentration formulation.

5. The stabilizing catheter of claim 4, wherein the high concentration formulation is greater than about 100 U/ml.

6. The stabilizing catheter of claim 1, wherein the one or more materials of the at least one layer includes materials selected from at least polytetrafluoroethane, saran (PVOC) polysulfone, glass, metal, derivatives of these materials, and mixtures of these materials.

7. The stabilizing catheter of claim 6, wherein the glass includes glass fibers.

8. The stabilizing catheter of claim 6, wherein the metal includes a braided metal.

9. The stabilizing catheter of claim 9, wherein the tubing includes at least two layers.

10. The stabilizing catheter of claim 9, wherein one layer includes materials selected from at least polytetrafluoroethane, saran (PVOC), polysulfide, glass, metal, derivatives of these materials, and mixtures of these materials.

11. The stabilizing catheter of claim 9, wherein one layer includes silicone, polyurethane, derivatives of these materials or mixtures of these materials.

12. The stabilizing catheter of claim 12, wherein the layer including silicone, polyurethane, derivatives of these materials or mixtures of these materials is the outer layer of the tubing.

13. The stabilizing catheter of claim 9, comprising an innermost layer that is formed from one or more hydrophilic protein compatible materials.

14. The stabilizing catheter of claim 13, wherein the hydrophilic protein compatible materials are selected from at least a polyethylene glycol, a polyurethane, a Genapol, a Tween, a Triton-X and a Brij, derivatives of these materials and mixtures of these materials.

15. The stabilizing catheter of claim 9, comprising three layers, an outer layer including a silicone material and a layer including materials selected from at least polytetrafluoroethane, saran (PVOC), polysulfone, glass, metal, derivatives of these materials, and mixtures of these materials, and an innermost layer that includes one or more hydrophilic insulin compatible materials.

16. The stabilizing catheter of claim 1, wherein the small molecules have a molecular weight of about 18 g/mole to about 500 g/mole.

17. The stabilizing catheter of claim 1, wherein the small molecules include neutral molecules, charged molecules, or mixtures of these molecules.

18. The stabilizing catheter of claim 17, wherein the charged molecules include metal ions.

19. The stabilizing catheter of claim 17, wherein the neutral molecules include at least phenol, phenolic derivatives, carbon dioxide, or mixtures of these molecules.

20. The stabilizing catheter of claim 19, wherein the stabilizing catheter reduces a diffusional flow of carbon dioxide into the tubing up to about 1000 fold as compared to the diffusional flow of carbon dioxide into a different tubing that is free of a stabilizing layer.

21. The stabilizing catheter of claim 20, wherein the stabilizing catheter reduces a diffusional flow of carbon dioxide into the tubing about 10-100 fold.

22. The stabilizing catheter of claim 19, wherein the stabilizing catheter reduces a diffusional flow of phenol, phenolic derivatives, or both, out from the tubing up to about 100 fold as compared to the diffusional flow of phenol, phenolic derivatives, or both, out from a different tubing that is free of a stabilizing layer.

23. The stabilizing catheter of claim 22, wherein the stabilizing catheter reduces a diffusional flow of carbon dioxide into the tubing about 2-20 fold.

24. The stabilizing catheter of claim 19, wherein the stabilizing catheter provides a diffusional barrier to phenol and phenolic derivatives such that the loss of phenol and phenolic derivatives through the tubing is less than about 5%, ±1%, at an protein drug infusion rate of about 20 U/day.

25. The stabilizing catheter of claim 6, where the layer of Teflon and/or saran is about 0.002 in to about 0.02 in (about 50 to about 500 microns).

26. The stabilizing catheter of claim 1, wherein the protein drug is an insulin analogue.

27. The stabilizing catheter of claim 26, wherein the insulin analogue is LISPRO.

28. An infusion system for protein drug delivery to a user, the infusion system comprising:
an infusion device housing;
at least one reservoir within the housing, wherein the reservoir is used for containing at least one protein for delivery to the user;
a drive mechanism within the housing; and
a stabilizing catheter having a distal end and a proximal end with the proximal end being connected to the reservoir, wherein the stabilizing catheter includes at least one layer, the at least one layer including one or more materials that reduce diffusion of small molecules through the tubing to provide a stabilizing layer, such that when the stabilizing catheter is used to deliver at least one protein, the protein drug formulation is stabilized as compared with a protein drug formulation delivered via a different tubing including one or more materials that are free of an effect that reduces diffusion of small molecules through the tubing.

29. The infusion system of claim 28, further including an exit tip connected to the distal end of the stabilizing catheter.

30. The infusion system of claim 28, wherein the stabilized protein drug is maintained in the tubing to substantially prevent occlusions from being formed during delivery of the protein drug.

31. The infusion system of claim 28, wherein the protein drug formulation is a high concentration insulin formulation.

32. The infusion system of claim 31, wherein the high concentration insulin formulation is greater than about 100 U/ml.

33. The infusion system of claim 28, wherein the insulin is an insulin analogue.

34. The infusion system of claim 33, wherein the insulin is LISPRO.

35. The infusion system of claim 28, wherein the one or more materials of the at least one material layer includes materials selected from at least polytetrafluoroethane, saran (PVOC), glass, metal, derivatives of these materials, and mixtures of these materials.

36. The infusion system of claim 35, wherein the glass material includes glass fibers.

37. The infusion system of claim 35, wherein the metal material includes a braided metal.

38. The infusion system of claim 28, wherein the stabilizing catheter includes more than one layer.

39. The infusion system of claim 35, wherein the stabilizing catheter further includes a layer comprising a silicone material.

40. The infusion system of claim 39, wherein the layer comprising silicone is an outer layer of the stabilizing catheter.

41. The pump system of claim 40, further including an inner layer including materials selected from at least polytetrafluoroethane, saran (PVOC), glass, metal, derivatives of these materials, and mixtures of these materials.

42. The infusion system of claim 28, wherein the stabilizing catheter includes two layers, an outer layer including a silicone and a layer including materials selected from at least polytetrafluoroethane, saran (PVOC), glass, metal, derivatives of these materials, and mixtures of these materials.

43. The infusion system of claim 28, wherein the one or more materials of the at least one layer of the stabilizing catheter includes at least one elastomer.

44. The infusion system of claim 28, further including at least a second layer, wherein the second layer forms an innermost layer and is formed from one or more hydrophilic insulin compatible materials.

45. The infusion system of claim 28, wherein the insulin compatible materials are selected from at least a polyethylene glycol, a polyurethane, a Genapol, a Tween, a Triton and a Brig, derivatives of these materials and mixtures of these materials.

46. The infusion system of claim 28, wherein the small molecules have a molecular weight of about 18 g/mole to about 500 g/mole.

47. The infusion system of claim 28, wherein the small molecules include neutral molecules, charged molecules, or mixtures of these molecules.

48. The infusion system of claim 47, wherein the neutral molecules include at least phenol, phenolic derivatives, carbon dioxide, or mixtures of these molecules.

49. The infusion system of claim 47, wherein the charged molecules include metal ions.

50. The infusion system of claim 48, wherein the stabilizing catheter reduces a diffusional flow of carbon dioxide into the tubing by approximately 10-100 fold as compared to the diffusional flow of carbon dioxide into a different tubing that is free of a stabilizing layer.

51. The infusion system of claim 48, wherein the stabilizing catheter reduces a diffusional flow of phenol, phenolic derivatives, or mixtures of these molecules, out from the tubing by approximately 2-20 fold as compared to the diffusional flow of phenol, phenolic derivatives, or mixtures of these molecules, out from a different tubing that is free of a stabilizing layer.

52. The infusion system of claim 28, wherein the stabilizing catheter provides a diffusional barrier to phenol, such that the loss of phenol through the tubing is less than about 5%, +/−1%, at an insulin infusion rate of about 20 U/day.

53. The infusion system of claim 35, where the layer of Teflon and/or saran (PVOC) is about 0.002 in to about 0.02 in (about 50 to about 500 microns).

54. A method of stabilizing an protein drug formulation in a drug delivery catheter, the method comprising:
providing a stabilizing catheter, wherein the stabilizing catheter includes at least one layer that includes one or more materials that reduce diffusion of small molecules through the tubing, such that when the stabilizing catheter is used to deliver the protein drug to a user, the protein drug is stabilized as compared with the protein drug delivered via a catheter that includes one or more materials that are free of an effect that reduces diffusion of small molecules through the catheter; and
flowing a fluid including the protein drug through the stabilizing catheter.

55. The method of claim 54, wherein the stabilized protein drug is maintained in the tubing to substantially prevent occlusions from being formed during delivery of the protein drug.

56. The method of claim 54, wherein the protein drug is a high concentration insulin formulation.

57. The method of claim 56, wherein the high concentration formulation is greater than about 100 U/ml.

58. The method of claim 56, wherein the insulin is a human analogue insulin.

59. The method of claim 58, wherein the insulin is LISPRO.

60. The method of claim 54, wherein the one or more materials of the at least one layer includes materials selected from at least polytetrafluoroethane, saran (PVOC), glass, a metal, derivatives of these of these materials, and mixtures of these materials.

61. The method of claim 54, wherein the stabilizing catheter includes more than one layer.

62. The method of claim 54, wherein the stabilizing catheter comprises two layers, an outer layer including a silicone material and an inner layer including materials selected from at least polytetrafluoroethane, saran (PVOC), glass, a metal, derivatives of these materials, and mixtures of these materials.

63. The method of claim 54, wherein the small molecules have a molecular weight of about 18 g/mole to about 300 g/mole.

64. The method of claim 54, wherein the small molecules include neutral molecules, charged molecules, or mixtures of these molecules

65. The method of claim 54, wherein the neutral molecules include at least phenol, phenolic derivatives, carbon dioxide, or mixtures of these molecules.

66. The method of claim 54, wherein the charged molecules include metal ions.

67. The method of claim 54, wherein the stabilizing catheter maintains body fluids surrounding an implantable stabilizing catheter by reducing the diffusional flow of small molecules out from the stabilizing catheter and into a body of the user.

68. A stabilizing catheter for protein drug delivery to a user, the stabilizing catheter comprising:
a tubing including at least one layer, wherein the at least one layer includes a stabilizing means that reduces the diffusion of small molecules through the stabilizing means, such that when the stabilizing means is used to deliver insulin, the protein drug formulation is stabilized as compared with the protein drug formulation delivered via a different tubing that includes one or more materials that are free of the effect that reduces diffusion of small molecules through the tubing.

69. The method of claim 68, wherein the stabilized protein drug is maintained in the tubing to substantially prevent occlusions or deposits from being formed during delivery.

70. A stabilizing catheter for use in protein delivery to a site within the body comprising:
a tubing including an interior surface;
a hydrophilic and mobile layer that is in affixed to the interior surface of the tubing, wherein as the protein traverses through the tubing and is in contact with the hydrophilic and mobile layer, the protein substantially remains in its biologically/pharmacologically active form for delivery to a site within the body as compared to the same tubing that does not contain a hydrophilic and mobile layer on its interior surface.

71. The stabilizing catheter of claim 70, wherein the protein is insulin.

72. The stabilizing catheter of claim 71, wherein the stabilizing substantially reduces site loss of insulin at the site of delivery within the body as compared to the same tubing that does not contain a hydrophilic and mobile layer on its interior surface.

73. A stabilizing catheter for use in protein delivery to a site within the body comprising:
a tubing that substantially reduces denaturation of the protein as it traverses through the tubing, thus maintaining the biologically/pharmacologically active form of the protein for delivery to the delivery site within the body.

74. A protein delivery tubing that maintains the biologically/pharmacologically active form of a protein drug for delivery to a delivery site within the body.

75. The protein delivery tubing of claim 74, wherein the delivery site is subcutaneous

76. The protein delivery tubing of claim 74, wherein the delivery site is intraperitoneal.

77. The protein delivery tubing of claim 74, wherein the protein drug is delivered via an external infusion drug delivery device

78. The protein delivery tubing of claim 74, wherein the protein drug is delivered via an internally implanted drug delivery device.

79. The protein delivery tubing of claim 74, wherein the delivery tubing includes a layer of a hydrophilic and mobile polymer affixed to the interior of the tubing.

80. The protein delivery tubing of claim 79, wherein the hydrophilic and mobile polymer includes polyethylene glycol.

81. A method of reducing site loss of a protein drug comprising:
maintaining a biologically/pharmacologically active form of the protein drug for delivery to a site within the body via a catheter attached to a protein drug infusion device.

82 The method of claim 81, wherein the biologically/pharmacologically active form of the protein drug is maintained by controlling for changes in a protein drug formulation as it traverses through the delivery catheter.

83. The method of claim 82, wherein the protein drug formulation is controlled for phenol and/or zinc loss.

84. The method of claim 81, wherein the resident time of the protein drug at a point within the catheter is reduced by reducing the catheter diameter.

85. A method of reducing site loss of a protein drug comprising:
providing a tubing with interior walls that form a surface;
providing a hydrophilic and mobile coating the surfaces of the interior walls of the tubing;
flowing a protein drug through the tubing to a desired site within the body; and
delivering the protein drug to the desired site within the body in a biologically/pharmaocologically active form.

86. The method of claim 85, further comprising providing a tubing that includes one or more materials that substantially prevent the diffusion of small molecules into and out from the tubing.
